(19) United States
(12) Patent Application Publication
(10) Pub. No.: US 2004/0266727 A1
(43) Pub. Date: Dec. 30, 2004

(54) Nutritional supplement having a unique soluble and insoluble fiber blend and method of making the same

(76) Inventor: Steve F. Brandon, Roanoke, TX (US)

Correspondence Address: BRYAN W. BOCKHOP, ESQ., ARNALL GOLDEN GREGORY LLP, 2800 ONE ATLANTA CENTER, 1201 WEST PEACHTREE STREET, ATLANTA, GA, 30309-3450, US

(21) Appl. No.: 10/877,300

(22) Filed: Jun. 25, 2004

Related U.S. Application Data
(60) Provisional application No. 60/482,417, filed on Jun. 25, 2003.

(51) Int. Cl. A61K 31/716; A61K 31/715

(52) U.S. Cl. 514/54; 514/57

(57) ABSTRACT

A nutritional supplement that includes a natural soluble fiber and a natural insoluble fiber is disclosed. The nutritional supplement includes an amount of a natural soluble fiber from 30% to 70% by weight, and an amount of a natural insoluble fiber from 30% to 70% by weight. The natural soluble fiber that is used is inulin, and the insoluble fiber that is used is cellulose. Xanthan gum is added in a negligible amount and serves as a stabilizer and to improve shelf life by helping to resist enzymatic degradation. A method of preparing a nutritional supplement is also disclosed. The insoluble fiber and the soluble fiber are blended to form a substantially homogenous, quickly dispersing powder blend. The powder blend can then be formed into a capsule, tablet, or wafer, or alternatively can be used as a food or drink additive.

### PRIORITY

This application claims priority to Provisional Application Ser. No. 60/482,417, filed on Jun. 25, 2003.

### BACKGROUND OF THE INVENTION

1. Field of the Invention

The present invention relates to a nutritional supplement having a blend of a natural soluble fiber and a natural insoluble fiber. More specifically, the nutritional supplement serves as a therapeutic dietary fiber supplement regimen to promote bowel regularity and a generally healthy well being within the digestive system. The present invention also relates to a method of making a nutritional supplement having a blend of soluble and insoluble fiber.

2. Description of the Prior Art

Approximately seventy million Americans suffer from digestive diseases or disorders, 15% of those on a daily basis. The health of the large intestinal wall and its microbial ecosystem are critical to gastrointestinal health. Further, in addition to serving as a primary site for water re-absorption, the colon is a major immune organ which functions-as a barrier against foreign materials from dietary or microbial origins from crossing into the internal body cavity. Through its fermentation, dietary fiber and its resultant short chain fatty acids (SCFAs) are important in protecting the health of the large intestinal wall and stimulating repair in a damaged colon.

It is well known that dietary soluble fiber, when part of a diet low in saturated fat and cholesterol, may reduce the risk of coronary heart disease, and that diets low in fat and rich in fiber-containing grain products, fruits and vegetables may reduce the risk of some types of cancer, including colon and breast cancer. It has also been demonstrated that several digestive health benefits are provided by a therapeutic dietary fiber regimen. Among those digestive maladies addressed by such therapeutic dietary fiber regimen are irritable bowel syndrome, diverticulosis, constipation, anal fissures, hemorrhoids, hiatus hernia, bowel polyps and bowel cancer. Some of these digestive maladies are addressed more effectively by soluble dietary fiber, others are addressed more effectively by insoluble dietary fiber.

There are two types of dietary fiber: insoluble fiber and soluble fiber. Sources of natural soluble fibers include flax seed, psyllium, pectins, and xanthan gum. Sources of natural insoluble fibers include cereal brans such as wheat, corn, barley, and rye oats. Cereal brans are further made up of cellulose, hemicellulose, and lignin. Cellulose is an insoluble fiber and is the major constituent of the cell wall of all plant cells. Many natural sources contain both soluble and insoluble fiber, such as fruits and vegetables. The choice of fibers, soluble or insoluble, in a dietary fiber product is essentially a matter of selecting which of two options produces the desired result most effectively and efficiently.

The primary physiological effects of dietary fiber are (1) gastric emptying and small intestine transit time, and (2) colonic transit time and large bowel functions due to fermentation by ceco-colonic microbial flora or bulking action. Physiological effects of dietary fibers differ in the small intestine and colon. Soluble fibers function to bind with fatty acids and prolong stomach emptying time to delay absorption and slow transit in the small intestine. Soluble fiber is also rapidly degraded by colonic bacteria, thereby influencing colonic function. Soluble fiber is fermented by a variety of anaerobic bacteria, resulting in an increase in bacterial biomass, an increase in fecal mass, and a change in introcolonic pH. Soluble fiber helps to regulate blood sugar and to lower total cholesterol and LDL cholesterol. Insoluble fibers function almost exclusively as bulking agents to provide increased fecal mass, promoting regular bowel movements, preventing constipation, and removing toxic waste through the colon in a shorter transit time. Insoluble fibers also control and balance the pH in the intestines, thereby keeping an optimal pH in the intestines to prevent microbes from producing cancer substances. All current commercially available soluble fibers form a gel when mixed with liquid, while insoluble fiber does not. Insoluble fiber passes through the intestines largely intact.

The recommended daily dietary fiber intake is between 20 and 30 grams (approximately one ounce) per day to maintain bowel regularity and general health. The National Academy of Science is considering establishing guidelines, Dietary Reference Intakes, which would raise the recommended level of dietary fiber intake to 35 grams per day. These guidelines for ingestion of dietary fiber indicate 25 grams per day of a mix of 30% soluble fiber and 70% insoluble fiber. However, the American Dietetic Association estimates the ‘typical’ daily diet consists of 8-14 grams of fiber. This shortage between the recommended daily fiber intake and the typical daily dietary intake is necessarily addressed by adding a fiber supplement to the diet.

There are a variety of fiber-based commercial products available to promote bowel regularity, using different fiber sources, which produce different physiological effects within the digestive system. Unifiber®, which is manufactured by Niche Pharmaceuticals, is an insoluble fiber made from highly refined plant cellulose, drawing water into the colon to increase fecal bulk and alter the pH level of the large intestine to interfere with microbial activity. Metamucil®, which is manufactured by The Procter and Gamble Company, is a soluble fiber made from psyllium husks, in which the fiber swells to provide bulk to form an easily eliminated stool. Citrucel®, which is manufactured by GlaxoSmithKline, is a soluble, indigestible fiber made from methylcellulose, a synthetic fiber which gels and adds bulk and texture to food as it passes through the digestive system. FiberCon®, which is manufactured by Whitehall-Robins, is a caplet containing Calcium Polycarbophil, a soluble, highly absorbent fiber, adding bulk and moisture to stools for easier elimination. FiberChoice®, which is manufactured by CNS, is a chewable tablet containing inulin, a soluble, prebiotic fiber, increasing stool bulk and moisture for easier elimination. Each of these products provides between 0.5 and 3.4 grams of fiber per dose.

There are several problems with the current fiber supplement products currently on the market. First, psyllium ferments in the large intestine, often causing excessive gas and cramping. Further, none of these products offers both soluble and insoluble fiber components, thereby requiring individuals to take multiple supplements to ensure that they are receiving both types of fiber as recommended. It has previously been difficult to manufacture a nutritional supplement that is made up of an insoluble fiber and a soluble fiber, due to the different properties that the two types of fiber exhibit when mixed with a liquid. When a soluble fiber and an insoluble fiber are blended together and combined with a liquid, the soluble fiber—insoluble fiber blend immediately forms an unappealing mass that is very unpleasant to ingest.

The present invention overcomes these problems and solves a need in the market by providing a nutritional supplement that provides the benefits of both soluble and insoluble fiber. The present invention further provides a nutritional supplement that includes soluble and insoluble fiber in a ratio that is commensurate with the recommended daily intake ratio. The present invention further provides a nutritional supplement that provides approximately 7 grams of fiber in one dose, which greatly increases the ability to make up for the gap between the typical dietary fiber intake and the recommended daily dietary fiber intake.

### SUMMARY OF THE INVENTION

The present invention includes the specific dietary fiber ingredients, soluble and insoluble, and formulation ratios to form a dietary fiber supplement which maximizes the intended physiological effects of a therapeutic dietary fiber regimen while minimizing, or eliminating, the adverse side effects inherent in many commercially available single-source soluble fiber formulations.

The present invention includes a combination soluble and insoluble dietary fiber therapeutic supplement that serves to address a broad spectrum of digestive issues. The incorporation of the specific soluble and insoluble dietary fibers of the present invention serves to provide prebiotic properties and functions, increase the bioavailablity of minerals, particularly calcium and magnesium, as well as other vitamins, reduce colonic pH to suppress microbial activity which produces carcinogens, stimulate bifidobacteria and suppress pathogenic bacteria, reduce liver toxins, carcinogens and food intolerances, provide immune system stimulation functions, and increase fecal bulk for easier elimination. The formulation of the present invention promotes bowel regularity and a generally healthy well being within the digestive system through the physiological effects of both soluble and insoluble fiber, which have previously been competing, or contradictory, systems. The formulation of the present invention serves as both a preventive agent, reducing the adverse potential of contracting digestive system maladies, as well as a proactive agent, increasing the beneficial potential of improving the functions of the digestive system, thereby improving the general well being.

The present invention also includes a method of mixing the ingredients to produce the formulation of a nutritional supplement.

In the present invention, a natural fiber blend serves as a therapeutic dietary fiber supplement regimen to promote bowel regularity and a generally healthy well being within the digestive system, the benefits and utility of each of the ingredients, individually and collectively, serve to achieve the objective of the present invention. The utility of insoluble dietary fiber, soluble dietary fiber, and a soluble, indigestible gum providing formulation stability and shelf life are combined in the present invention to achieve a synergy unique to therapeutic dietary fiber regimen.

The resulting material, fabricated in the art of the present invention, is a formulation of a natural soluble dietary fiber, a natural insoluble dietary fiber, and a dispersing agent which material, when blended in the preferred amount with liquids such as water, juice, dairy milk, soy milk, rice milk or other beverage, or with soft foods such as cereals, applesauce, mashed potatoes or similar foods, provides a more effective and efficient therapeutic dietary fiber regimen than provided by a single-type, soluble or insoluble, source of dietary fiber by virtue of its increased beneficial microbial activity, enhanced water absorption, effective stool softening, increased rate of fecal transit and improved gastrointestinal health and general well being. The resulting material may also be blended as a singular recipe ingredient in the preferred amount and combined with other solid and liquid ingredients for cooked or baked foods, to serve as a significant source of therapeutic dietary fiber. The resulting material may also be fabricated as a tablet, capsule, chewable tablet, chewable wafer or cookie to serve as a significant source of therapeutic dietary fiber. Just one daily serving offers beneficial gastrointestinal effects for the intended purpose of the present invention, although more than one serving may be taken for an individual's particular needs.

These and other aspects of the invention will become apparent from the following description of the preferred embodiments. As would be obvious to one skilled in the art, many variations and modifications of the invention may be affected without departing from the spirit and scope of the disclosure.

### DETAILED DESCRIPTION OF THE INVENTION

A preferred embodiment of the invention is now described in detail. As used in the description herein and throughout the claims, the following terms take the meanings explicitly associated herein, unless the context clearly dictates otherwise: the meaning of “a,” “an,” and “the” includes plural reference, the meaning of “in” includes “in” and “on.”

The present invention combines a soluble fiber and an insoluble fiber into a high fiber nutritional supplement. The nutritional supplement of the present invention preferably uses natural soluble fiber and natural insoluble fiber. It is more preferable to use inulin as the source of natural soluble fiber for the nutritional supplement of the present invention. It is preferable to use cellulose as the source of natural insoluble fiber for the nutritional supplement of the present invention. The nutritional supplement of the present invention preferably includes a soluble fiber component in the amount of 30-70% by weight and an insoluble fiber component in the amount of 30-70% by weight. One illustrative embodiment of the invention includes a soluble fiber component in the amount of 49% by weight and an insoluble fiber component in the amount of 49% by weight. Another illustrative embodiment of the invention includes a second source of soluble fiber that functions as a stabilizer and improves formulation shelf life in the amount of 1-5% by weight, more preferably 2% by weight. The second source of soluble fiber may include xanthan gum.

Inulin, which is sold under the trademark Frutafit®, and is manufactured by Imperial Sensus, LLC, is a natural functional food ingredient extracted from the root of the chicory plant, Cichorium intybus, and is a readily soluble dietary fiber having neutral taste, odor and color. Inulin is also referred to as ‘polyfructose’, ‘fructan’, or ‘fructooligosaccharides (FOS)’. Inulin is a polydisperse GFn molecule built as a linear chain of fructose units by means of β (2-1) bonds, having one terminal glucose molecule at each end, and provides exceptional gelling properties and a high water retention capacity. Among its nutritional properties, inulin is low in calories (1.5 kcal/g) insofar as it is not digested, reduces colonic pH, has a glycemic index of zero, having no influence on blood glucose nor stimulation of insulin excretion, and stimulates bifidus, inhibiting harmful bacteria and reducing toxins and carcinogens, while activating the immune system and providing for the synthesis of vitamins and improving mineral absorption.

Inulin is not digested, reaching the colon intact, to be hydrolyzed and fermented by beneficial caecal bacteria residing in the colon, producing short-chain fatty acids such as acetic, propionic and butyric acids. Acetic and propionate acids are used by the liver for energy production, while butyric acid has demonstrated cancer-preventing properties within the intestine. Inulin functions physiologically as a soluble dietary fiber but, unlike other soluble dietary fibers, inulin does not bind with metal and does not inhibit mineral absorption or interfere with the absorption of medications. Short-chain fatty acids produced from inulin fermentation decrease intestinal pH and stimulate proliferation of colon epithelial cells, enhancing the absorption of certain cations (particularly Ca⁺², as well as Mg⁺², Cu⁺², An⁺² and Fe⁺²).

Inulin provides significant intestinal health, digestion and well being functions, including enhanced fecal bulk and weight, increased fecal frequency, increased colon mucosal weight, reduced fecal pH, toxic and carcinogenic metabolites, reduction in serum lipids, reduction in blood cholesterol, increased HLD/LDL ratio, reduction in glycemic response, amelioration in the derangements of carbohydrate and lipid metabolism, modification of composition and rate of production of secondary bile acids, improved mineral absorption, and modification of fecal microflora (Bifidus stimulation).

Because inulin is not digested, arriving unchanged to the colon, it also serves as a prebiotic. Beneficial bacteria, notably bifidobacteria and lactobacilli, use inulin for growth and proliferation. Most pathogenic and putrefactive bacteria cannot break the bonds holding inulin together. Multiplying beneficial bacteria reduce the pH level in the colon, making the environment uninhabitable for potentially harmful bacteria such as E. coli, clostridia, Veillonella and Klebsiella. The proliferation of beneficial bacteria provides significant health effects, including enhancing digestion and improves lactose intolerance, promoting the recycling of compounds such as estrogen, synthesizing vitamins, especially B-group vitamins, producing immune-stimulating compounds, inhibiting the growth of harmful bacteria, reducing the production of toxins and carcinogens, restoring normal intestinal bacteria during antibiotic therapy, and reducing the potential for several pathologies commonly associated with higher numbers of pathogenic intestinal bacteria such as autoimmune illnesses such as ankylosing spondylitis and rheumatoid arthritis, certain cancers, yeast overgrowth, vaginitis, urinary tract infections, cirrhosis of the liver, food poisoning, antibiotic-associated diarrhea, inflammatory bowel diseases such as ulcerative colitis and Crohn's disease, necrotizing entercolitis and ileocecitis, food allergy and intolerance, intestinal gas and bloating, and irritable bowel syndrome.

The primary benefits of insoluble fiber in the digestive system include its ability to increase fecal weight and accelerate colonic transit. Cellulose, specifically as an ultra-fine powder, is an insoluble fiber that draws water into the colon to increase fecal bulk, softening the stool for easier elimination. Additionally, insoluble cellulose dilutes the concentration of potential carcinogens, reducing their exposure to the intestinal wall. Powered cellulose may bind liquids by as much as ten times its weight in water; however, by virtue of being inert, it does not interfere with the absorption of medications, minerals or vitamins.

Xanthan Gum is a soluble, readily fermentable fiber. Xanthan Gum is a stabilizer, retains water, improves formulation shelf life and is highly resistant to enzymatic degradation. Xanthan Gum is a long chain polysaccharide, a polymer made of sugar subunits, like starch or cellulose. Xanthan Gum is composed of glucose, mannose and glucuronic acid, linked to form a strand of cellulose with trisaccharide side chains branching off every fifth cellulose sugar. Xanthan Gum has the consistency of cornstarch and is both soluble and indigestible. Xanthan Gum is produced by fermenting corn sugar with Xanthomonas campestris bacteria.

It is shown by the present invention that inulin can be blended with an insoluble fiber to form a nutritional supplement. To achieve the desired results of the present invention, specifically promoting bowel regularity and a generally healthy well being within the digestive system through the physiological effects of merging otherwise competing, or contradictory, systems, a limited, acceptable formulation is presented:

| Ingredient | Percentage by Weight |
|---|---|
| Powdered Cellulose (insoluble) | 30-70 |
| Inulin (soluble) | 70-30 |
| Xanthan Gum | 01-05 |

With the preferred embodiment of the unique formulation of the present invention:

| Ingredient | Percentage by Weight |
|---|---|
| Powdered Cellulose (insoluble) | 49 |
| Inulin (soluble) | 49 |
| Xanthan Gum | 02 |

The formulation of the present invention provides an optimal therapeutic dietary fiber regimen with the balanced combination of soluble and insoluble fibers and dispersing agents.

The nutritional supplement of the present invention is formulated by first determining the total desired weight amount of the unique formulation of the present invention. The total required weight amount of each of the ingredients of the unique formulation of the present invention is then determined by ingredient ratio of the total desired weight amount. The measured amounts of the individual ingredients are combined in a mixing container and thoroughly blended, achieving the purpose of producing a uniform, quickly dispersing powder of the present invention. The measured amounts of the individual ingredients may be combined and agglomerated, achieving the purpose of producing a uniform, quickly dispersing powder of the present invention. No unique or extraordinary mixing or blending equipment is required to obtain the desired nutritional supplement.

In one illustrative embodiment of the invention, powdered cellulose #200 NF, 49% by weight, is mixed with Frutafit®, 49% by weight, and xanthan gum, 2% by weight, in a sterile stainless steel drum and blended for two to six minutes under air heat at 60 to 150 degrees Celcius. The resulting initial blend is sprayed with distilled water at a rate of 0.5 to 2 ml/minute for 30-40 minutes to form a second interim blend. This second interim blend is air dryed at 60 to 150 degrees Celsius for 3 to 4 hours, until the moisture content is between 2 and 8% to form a third interim product. This third interim product is sifted through a standard #20 mesh screen to form a substantially homogenous powder blend having an extra fine particle size. When approximately 7 grams of the substantially homogenous powder blend is added to approximately 8 ounces of water and stirred for approximately 30 seconds and then passed through a standard #20 mesh screen, no lumps, gels, or gritty product are retained by the screen.

The substantially homogenous powder blend can be sold directly to consumers for use as a nutritional supplement, or it can be formed into tablets, including chewable tablets, capsules, chewable wafers, or cookies, using methods well known to those of skill in the art, and then sold to consumers for use as a nutritional supplement.

To achieve the results indicated in the present invention, the preferred dosage is suggested to be one-quarter ounce (7.0875 grams (1.8 teaspoons)) of the substantially homogenous powder blend of the present invention, blended with six ounces of liquid, with soft food, or as a singular recipe ingredient for cooked or baked foods, providing 6.237 grams of dietary fiber.

Per Serving Nutrition Information:

| | | |
|---|---|---|
| Serving size | 7.0875 | grams (0.25 ounces) |
| Calories | 14.44 | kcal |
| Carbohydrates | 3.61 | grams |
| Dietary Fiber | 7.09 | grams |

All three components (inulin, Xanthan Gum and powdered cellulose) are considered dietary fiber, but only the inulin and Xanthan Gum are counted as total carbohydrates, per the 1990 NLEA and 2 CFR 101.9. Calculations for pre-serving nutritional information, based on 7.0875 grams (0.25 ounces) are:

Total carbohydrates: 51% of 7.0875 g=3.61 g

Calories: 4 kcal/gram of carbohydrates, per 21 CFR 101.9

Dietary fiber: 7.09 grams (rounding from 7.0875)

The above-described embodiments are given as illustrative examples only. It will be readily appreciated that many deviations may be made from the specific embodiments disclosed in this specification without departing from the invention. Accordingly, the scope of the invention is to be determined by the claims below rather than being limited to the specifically described embodiments above.

What is claimed is:

1. A nutritional supplement consisting essentially of:
a. a natural soluble fiber; and
b. a natural insoluble fiber.

2. The nutritional supplement of claim 1, comprising between about 30% and about 70% by weight of the insoluble fiber.

3. The nutritional supplement of claim 2, comprising about 49% by weight of the insoluble fiber.

4. The nutritional supplement of claim 1, comprising between about 30% and about 70% by weight of the soluble fiber.

5. The nutritional supplement of claim 4, comprising about 49% by weight of the soluble fiber.

6. The nutritional supplement of claim 1, comprising a dietary fiber content of between about 5 grams and about 9 grams.

7. The nutritional supplement of claim 6, comprising a dietary fiber content of about 7 grams.

8. The nutritional supplement of claim 1, wherein the soluble fiber is inulin.

9. The nutritional supplement of claim 1, wherein the insoluble fiber is cellulose.

10. The nutritional supplement of claim 1, further comprising a second soluble fiber between about 1% and about 5% by weight.

11. The nutritional supplement of claim 10, further comprising a second soluble fiber of about 2% by weight.

12. The nutritional supplement of claim 11, wherein the second soluble fiber is xanthan gum.

13. The nutritional supplement of claim 1, wherein the nutritional supplement is in powder form.

14. The nutritional supplement of claim 1, wherein the nutritional supplement is in tablet form.

15. The nutritional supplement of claim 14, wherein the nutritional supplement is a chewable tablet.

16. The nutritional supplement of claim 1, wherein the nutritional supplement is in capsule form.

17. The nutritional supplement of claim 1, wherein the nutritional supplement is a chewable wafer.

18. A nutritional supplement consisting essentially of between about 30% and about 70% by weight of inulin, between about 30% and about 70% by weight of cellulose, and between about 1% and about 5% by weight of xanthan gum.

19. A method of formulating a nutritional supplement, comprising the steps of:
a. combining a predetermined amount of an insoluble fiber with a predetermined amount of a soluble fiber; and
b. mixing the insoluble fiber and the soluble fiber to produce a substantially homogeneous powder blend.

20. The method of claim 19, further comprising the step of forming the substantially homogeneous powder blend into a tablet.

21. The method of claim 19, further comprising the step of forming the substantially homogeneous powder blend into a caplet.

22. The method of claim 19, further comprising the step of forming the substantially homogeneous powder blend into a chewable wafer.

23. The method of claim 19, wherein the predetermined amount of the insoluble fiber is between about 30% and about 70% by weight of the substantially homogeneous powder blend.

24. The method of claim 23, wherein the predetermined amount of the insoluble fiber is about 49% by weight of the substantially homogeneous powder blend.

25. The method of claim 19, wherein the predetermined amount of the soluble fiber is between about 30% and about 70% by weight of the substantially homogeneous powder blend.

26. The method of claim 25, wherein the predetermined amount of the insoluble fiber is about 49% by weight of the substantially homogeneous powder blend.

27. The method of claim 19, wherein the nutritional supplement has a dietary fiber content of between about 5 grams and about 9 grams.

28. The method of claim 27, wherein the nutritional supplement has a dietary fiber content of about 7 grams.

29. The method of claim 19, wherein the soluble fiber is inulin.

30. The method of claim 19, wherein the insoluble fiber is cellulose.

31. The method of claim 19, further comprising the step of mixing a second soluble fiber with the soluble fiber and the insoluble fiber.

32. The method of claim 31, wherein the desired amount of the second soluble fiber is between about 1% and about 5% by weight of the substantially homogeneous powder blend.

33. The method of claim 32, wherein the desired amount of the second soluble fiber is about 2% by weight of the substantially homogeneous powder blend.

34. The method of claim 31, wherein the second soluble fiber is xanthan gum.

35. The method of claim 19, further comprising the step of forming the substantially homogenous powder blend into a tablet.

36. The method of claim 19, further comprising the step of forming the substantially homogenous powder blend into a capsule.

37. The method of claim 19, further comprising the step of forming the substantially homogenous powder blend into a chewable wafer.

38. A method of formulating a nutritional supplement, comprising the steps of:
a. combining inulin in an amount between about 30% and about 70% by weight with cellulose in an amount between about 30% and about 70% by weight and xanthan gum in an amount between about 1% and 5% by weight; and
b. mixing the inulin, the cellulose and the xanthan gum to produce a substantially homogeneous powder blend.
